(19) United States
(12) Patent Application Publication
(10) Pub. No.: US 2026/0102288 A1
(43) Pub. Date: Apr. 16, 2026

(54) SYSTEMS, APPARATUSES, AND METHODS FOR CREATING TISSUE INTERFACES

(71) Applicant: Solventum Intellectual Properties Company, Maplewood, MN (US)

(72) Inventors: Benjamin A. PRATT, Poole (GB); Timothy Mark ROBINSON, Shillingstone (GB); Thomas Alan EDWARDS, Hampshire (GB); Robert HOWARD, Westmeath (GB)

(21) Appl. No.: 19/111,716

(22) PCT Filed: Sep. 7, 2023
(86) PCT No.: PCT/IB2023/058878

§ 371 Date: Mar. 13, 2025

Related U.S. Application Data
(60) Provisional application No. 63/408,315, filed on Sep. 20, 2022.

(51) Int. Cl. A61F 13/00; A61B 5/00; A61F 13/05; B33Y 10/00; B33Y 30/00; B33Y 40/20; B33Y 50/00; B33Y 80/00

(52) U.S. Cl. CPC A61F 13/00987; A61B 5/0064; A61F 13/05; B33Y 50/00; B33Y 80/00; B33Y 10/00; B33Y 30/00; B33Y 40/20

(57) ABSTRACT

A system for creating a tissue interface. The system includes a scanner, at least one processor, and a memory. The scanner is configured to scan a tissue site and generate a signal corresponding to a topography of the tissue site. The at least one processor is configured to receive the signal corresponding to the topography of the tissue site and to receive user input at a user interface identifying a therapy to be applied at the tissue site. The memory is coupled to the at least one processor and is configured to store instructions that when executed by the at least one processor in response to receiving the signal corresponding to the topography of the tissue site cause the system to transform the signal corresponding to the topography of the tissue site into a model site surface and generate a model tissue interface.

### CROSS-REFERENCE TO RELATED APPLICATIONS

This application is a U.S. National Stage Entry of PCT International Application No. PCT/IB2023/058878, filed on Sep. 7, 2023, which claims the benefit of priority to U.S. Provisional Application No. 63/408,315, filed on Sep. 20, 2022, which is incorporated herein by reference in its entirety.

### TECHNICAL FIELD

The invention set forth in the appended claims relates generally to tissue treatment systems and more particularly, but without limitation, to systems, apparatuses, and methods of creating tissue interfaces.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

While the clinical benefits of negative-pressure therapy are widely known, improvements to therapy systems, components, and processes may benefit healthcare providers and patients.

### BRIEF SUMMARY

New and useful systems, apparatuses, and methods for creating customizable wound fillers for use in a negative-pressure therapy environment are set forth in the appended claims. Illustrative embodiments are also provided to enable a person skilled in the art to make and use the claimed subject matter.

For example, in some embodiments, a system for creating a tissue interface is described. The system can include a scanner, at least one processor, and a memory. The scanner can be configured to scan a tissue site and generate a signal corresponding to a topography of the tissue site. The at least one processor can be configured to receive the signal corresponding to the topography of the tissue site and to receive user input at a user interface identifying a therapy to be applied at the tissue site. The memory can be coupled to the at least one processor and can be configured to store instructions that when executed by the at least one processor in response to receiving the signal corresponding to the topography of the tissue site cause the system to transform the signal corresponding to the topography of the tissue site into a model site surface and generate a model tissue interface based on the model site surface and the therapy to be applied to the tissue site.

In some example embodiments, the instructions that when executed by the at least one processor can cause the system to create a tissue interface based on the model tissue interface.

In some example embodiments, creating the tissue interface based on the model tissue interface can include printing the tissue interface using fused deposition modeling. In some example embodiments, printing the tissue interface further includes depositing a plurality of bioabsorbable fibers in a plurality of layers. In some example embodiments, creating the tissue interface based on the model tissue interface can further include removing printing support material from the tissue interface. In some example embodiments, removing the printing support material can include soaking the tissue interface in a bath. In some example embodiments, soaking the tissue interface in a bath can include sterilizing the tissue interface.

In some example embodiments, creating the tissue interface based on the model tissue interface can include extruding a bioabsorbable material into a coagulation bath.

In some example embodiments, creating the tissue interface based on the model tissue interface can include printing the tissue interface using stereolithography. In some example embodiments, creating the tissue interface based on the model tissue interface can further include curing and sterilizing the tissue interface. In some example embodiments, curing and sterilizing the tissue interface can include exposing the tissue interface to an ultraviolet (UV) electromagnetic radiation source.

In some example embodiments, creating the tissue interface based on the model tissue interface can include stereolithography monochrome liquid crystal display (LCD) printing. In some example embodiments, creating the tissue interface based on the model tissue interface can further include curing and sterilizing the tissue interface. In some example embodiments, curing and sterilizing the tissue interface can include exposing the tissue interface to at least one UV LED.

In some example embodiments, creating the tissue interface based on the model tissue interface can include laser cutting the tissue interface to a predetermined size and shape based on the model tissue interface.

In some example embodiments, the instructions that when executed by the at least one processor can cause the system to create a template based on the model tissue interface. The template can be configured to enable a user to customize a tissue interface to fit the tissue site.

In some example embodiments, the memory can store an image database that can include images and information associated with different tissue sites. In some example embodiments, generating the model tissue interface based on the model site surface and the therapy to be applied to the tissue site can include comparing the model site surface and the therapy to be applied to the tissue site to the images and information in the image database.

Also described herein is a method of creating a tissue interface. The method can include scanning a tissue site to create at least one scanned image, transforming the at least one scanned image into a model site surface, identifying a therapy to be applied to the tissue site, and creating the tissue interface based on the model site surface and the therapy.

In some example embodiments, scanning the tissue site can include imaging the tissue site with laser imaging, detection, and ranging (LIDAR) technology. In some example embodiments, scanning the tissue site can further include imaging the tissue site with an ultrasound device configured to capture sub-surface features.

In some example embodiments, the at least one scanned image can include a three-dimensional (3-D) model of the tissue site.

In some example embodiments, the method can further include analyzing the at least one scanned image. If the at least one scanned image does not include a complete surface, the method can further include generating an indicator to re-scan the tissue site.

In some example embodiments, identifying the therapy to be applied to the tissue site can include identifying at least one of hydration, oxygen, bolstering, and closure.

In some example embodiments, the method can further include packaging the tissue interface.

Also described herein is a tissue interface. The tissue interface can include an outer section and at least one inner section. The outer section can have a density configured to prevent tissue ingrowth. The at least one inner section can be surrounded by the outer section. The at least one inner section can have an internal web and a density less than the density of the outer section.

In some example embodiments, the at least one inner section can include at least one coil.

In some example embodiments, the at least one inner section can include at least one wave.

In some example embodiments, the at least one inner section can be configured to allow the tissue interface to collapse in a vertical direction and a lateral direction.

In some example embodiments, at least one of the outer section and the at least one inner section can include at least one bioabsorbable polymer. In some example embodiments, the bioabsorbable polymer can include at least one of polylactic acid, polylactides, polycaprolactone, polyglycolic acid, polyglycolides, polydioxanone, and poly(glycerolsebacate).

In some example embodiments, the tissue interface can be configured to be positioned at a tissue site for more than three days.

Also described herein is a tissue interface including a contact layer and a plurality of bioabsorbable hooks. The contact layer can be configured to be positioned proximate to a tissue site. The contact layer can be configured to distribute fluid across the tissue site. The plurality of bioabsorbable hooks can extend from a surface of the contact layer. The plurality of bioabsorbable hooks can be configured to engage the tissue site and can be breakable to facilitate removal of the tissue interface from the tissue site.

In some example embodiments, the contact layer can include a woven sheet. In some example embodiments, the contact layer can include a perforated sheet.

Also described herein is a tissue interface including a support layer and a transition layer. The support layer can be configured to be positioned proximate to a tissue site. The transition layer can be coupled to the support layer. The transition layer can have a dimension that can be configured to transition between a first value and a second value in response to at least one of a change in temperature or exposure to a liquid.

In some example embodiments, the support layer can be configured to bend which may form a mesh to manifold pressure through the tissue interface when the dimension is transitioned between the first value and the second value. In some embodiments, the support layer can have a first rigidity and the transition layer can have a second rigidity. The first rigidity can be different from the second rigidity.

Also described herein is a tissue interface that can be produced by 3-D printing a bioabsorbable wound filler into a bundle. The bundle can be a controlled arrangement of the bioabsorbable wound filler that can allow negative pressure to be communicated through the tissue interface.

Also described herein is a tissue interface that can be produced by 3-D printing a geometric mesh. The geometric mesh can be configured to provide support to the tissue interface and can enable pressure manifolding through the tissue interface.

In some example embodiments, the geometric mesh can be formed from truncated octahedrons.

Also described herein is a tissue interface that can be produced by 3-D printing a first plurality of fibers in a first linear orientation and 3-D printing at least a second plurality of fibers onto the first plurality of fibers. The second plurality of fibers can be printed in a second linear orientation. The second linear orientation can be perpendicular to the first linear orientation.

In some example embodiments, the tissue interface can be between about 5 millimeters and about 20 millimeters thick.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a block diagram of an example embodiment of a system that can create a tissue interface in accordance with this specification;

FIG. 2 is a flow chart illustrating exemplary operations that may be associated with some embodiments of the system of FIG. 1;

FIG. 3 is a perspective view of a scanner scanning a tissue site including a wound;

FIG. 4 is a schematic representation of a user interface displaying of a scanned image of the tissue site of FIG. 3;

FIG. 5 is a schematic representation of the user interface displaying another scanned image of the tissue site of FIG. 3;

FIG. 6 is a side view of an exterior surface of a model site surface generated from the scanned image of FIG. 4;

FIG. 7 is a perspective view of an interior surface of the model site surface generated from the scanned image of FIG. 4;

FIG. 8 is a perspective view of a model tissue interface generated from the model site surface of FIG. 6 and FIG. 7;

FIG. 9 is a perspective view of a tissue interface created based on the wound;

FIG. 10 is a cross-sectional view of the tissue interface of FIG. 9 taken along line 10-10 of FIG. 9;

FIG. 11 is a cross-sectional view of the tissue interface of FIG. 9 including a connection aperture taken along line 10-10 of FIG. 9;

FIG. 12 is a cross sectional view of an example embodiment of a tissue interface that may be configured to collapse horizontally;

FIG. 13 is a cross sectional view of another example embodiment of a tissue interface that may be configured to collapse vertically;

FIG. 14 is a cross sectional view of another example embodiment of a tissue interface that may be configured to collapse in multiple directions;

FIG. 15 is a cross sectional view of another example embodiment of a tissue interface that may be configured to collapse vertically;

FIG. 16 is a perspective view of an example embodiment of a tissue interface with a plurality of collapsible portions;

FIG. 17 is a cross-sectional view of the tissue interface of FIG. 16 taken along line 17-17 of FIG. 16;

FIG. 18 is a cross-sectional view of the tissue interface of FIG. 17 being compressed horizontally and vertically;

FIG. 19 is a cross-sectional view of another embodiment of the tissue interface of FIG. 16 taken along line 17-17 of FIG. 16;

FIG. 20 is a cross-sectional view of another embodiment of the tissue interface of FIG. 16 taken along line 17-17 of FIG. 16;

FIG. 21 is a perspective view of an embodiment of a tissue interface including a bundle of filaments;

FIG. 22 is a perspective view of an embodiment of a tissue interface with a plurality of hooks configured to engage a tissue site;

FIG. 23 is a side view of the tissue interface of FIG. 22 applied to the tissue site;

FIG. 24 is a side view of the tissue interface of FIG. 23 being removed from the tissue site;

FIG. 25 is a side view of an embodiment of a tissue interface configured to shrink upon exposure to a change in temperature or to a liquid;

FIG. 26 is a side view of the tissue interface of FIG. 25 after being exposed to either a change in temperature or to a liquid;

FIG. 27 is an embodiment of a tissue interface with a truncated octahedron shape;

FIG. 28 is a top view of an embodiment of a tissue interface with a lattice structure; and

FIG. 29 is a perspective view of the tissue interface of FIG. 28 in a rolled configuration.

### DESCRIPTION OF EXAMPLE EMBODIMENTS

The following description of example embodiments provides information that enables a person skilled in the art to make and use the subject matter set forth in the appended claims, but it may omit certain details already well-known in the art. The following detailed description is, therefore, to be taken as illustrative and not limiting.

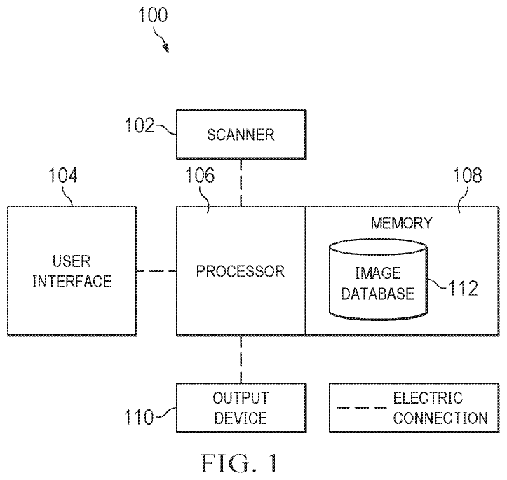

FIG. 1 is a block diagram of a system 100 that can be used to create a tissue interface for use with treating a tissue site. In some embodiments, the tissue site may be treated with negative pressure therapy. In some embodiments, the tissue interface can be generally adapted to partially or fully contact the tissue site. The tissue interface may be an element of a dressing that may be used to treat the tissue site. The tissue interface may take many forms, and may have many sizes, shapes, or thicknesses, depending on a variety of factors, such as the type of treatment being implemented or the nature and size of a tissue site. For example, the size and shape of the tissue interface may be adapted to the contours of deep and irregular shaped tissue sites. Any or all of the surfaces of the tissue interface may have an uneven, coarse, or jagged profile.

In some embodiments, the tissue interface may comprise or consist essentially of a manifold. A manifold in this context may comprise or consist essentially of an apparatus for collecting or distributing fluid across the tissue interface under pressure. For example, a manifold may be adapted to receive negative pressure from a source and distribute negative pressure through multiple apertures across the tissue interface, which may have the effect of collecting fluid from across a tissue site and drawing the fluid toward the source. In some embodiments, the fluid path may be reversed, or a secondary fluid path may be provided to facilitate delivering fluid across a tissue site.

In some illustrative embodiments, a manifold may comprise a plurality of pathways, which can be interconnected to improve distribution or collection of fluids. In some illustrative embodiments, a manifold may comprise or consist essentially of a porous material having interconnected fluid pathways. Examples of suitable porous material that can be adapted to form interconnected fluid pathways (e.g., channels) may include cellular foam, including open-cell foam such as reticulated foam; porous tissue collections; and other porous material such as gauze or felted mat that generally include pores, edges, and/or walls. Liquids, gels, and other foams may also include or be cured to include apertures and fluid pathways. In some embodiments, a manifold may additionally or alternatively comprise projections that form interconnected fluid pathways. For example, a manifold may be molded to provide surface projections that define interconnected fluid pathways.

In some embodiments, the tissue interface may comprise or consist essentially of reticulated foam having pore sizes and free volume that may vary according to needs of a prescribed therapy. For example, reticulated foam having a free volume of at least 90% may be suitable for many therapy applications, and foam having an average pore size in a range of 400-600 microns (40-50 pores per inch) may be particularly suitable for some types of therapy. The tensile strength of the tissue interface may also vary according to needs of a prescribed therapy. The 25% compression load deflection of the tissue interface may be at least 0.35 pounds per square inch, and the 65% compression load deflection may be at least 0.43 pounds per square inch. In some embodiments, the tensile strength of the tissue interface may be at least 10 pounds per square inch. The tissue interface may have a tear strength of at least 2.5 pounds per inch. In some embodiments, the tissue interface may be foam comprised of polyols such as polyester or polyether, isocyanate such as toluene diisocyanate, and polymerization modifiers such as amines and tin compounds. In some examples, the tissue interface may be reticulated polyurethane foam such as found in GRANUFOAM™ dressing or V.A.C. VERAFLO™ dressing, both available from 3M Company.

The thickness of the tissue interface may also vary according to needs of a prescribed therapy. For example, the thickness of the tissue interface may be decreased to reduce tension on peripheral tissue. The thickness of the tissue interface can also affect the conformability of the tissue interface. In some embodiments, a thickness in a range of about 5 millimeters to 10 millimeters may be suitable.

The tissue interface may be configured to facilitate a desired therapy. For example, the tissue interface may be a bolster. A tissue interface configured as a bolster may provide support to a tissue site. In some embodiments, the tissue interface may be configured to facilitate closure. The tissue interface may be considered an all-around closure device that may facilitate closure both vertically and horizontally. The tissue interface may be considered a longitudinal closure device that may be configured to facilitate horizontal closure of the tissue site. The tissue interface may be a depth reduction tissue interface that may be configured to facilitate vertical closure of the tissue site. In some embodiments, the tissue interface may be configured to facilitate hydration therapy and/or oxygen therapy. For example, the tissue interface may be designed to include pathways to facilitate movement of gasses and/or liquids through the tissue interface to reach the tissue site.

The tissue interface may be either hydrophobic or hydrophilic. In an example in which the tissue interface may be hydrophilic, the tissue interface may also wick fluid away from a tissue site, while continuing to distribute negative pressure to the tissue site. The wicking properties of the tissue interface may draw fluid away from a tissue site by capillary flow or other wicking mechanisms. An example of a hydrophilic material that may be suitable is a polyvinyl alcohol, open-cell foam such as V.A.C. WHITEFOAM™ dressing available from 3M Company. Other hydrophilic foams may include those made from polyether. Other foams that may exhibit hydrophilic characteristics include hydrophobic foams that have been treated or coated to provide hydrophilicity.

In some embodiments, the tissue interface may be constructed from bioresorbable materials such as bioabsorbable polymers. Suitable bioresorbable materials may include, without limitation, polylactic acid or polylactides (PLA), polycaprolactone (PCL), polyglycolic acid or polyglycolides (PGA), polydioxanone (PDO); poly(glycerolsebacate) (PGS). Any of the above listed materials may be copolymerized or blended with each other as well as with glycerin and/or polyethylene glycols (PEG) to form a tissue interface of a desired stiffness, elasticity, strength, and bio absorption time. In some embodiments, the tissue interface may be a polymeric blend of polylactic acid (PLA) and polyglycolic acid (PGA). The polymeric blend may also include, without limitation, polycarbonates, polyfumarates, and caprolactone. In some embodiments, the tissue interface may alternatively or additionally include alginates which are not absorbable and/or chitosan-based materials. In some embodiments, the plastic material of the tissue interface may be foamed as the tissue interface is formed to enhance stiffness and to control an amount of plastic used to create the tissue interface. Additionally or alternatively, the tissue interface may be formed from coextruded filaments. In some embodiments, the coextruded filaments may have heat and/or wet shrink properties that allow the tissue interface to shrink when exposed to liquid and/or heat.

The tissue interface may further serve as a scaffold for new cell-growth, or a scaffold material may be used in conjunction with the tissue interface to promote cell-growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials.

In some embodiments, the system 100 may include a scanner 102, a user interface 104, at least one processor, such as a processor 106, at least one memory, such as a memory 108, and an output device 110. The scanner 102 of the system 100 may be configured to scan an object to produce a signal corresponding to a topography of the object. In some embodiments, the scanner 102 may be configured to scan a tissue site to generate a signal corresponding to a topography of the tissue site. The topography of the tissue site may be a three-dimensional (3D) view of the tissue site. The scanner 102 may be a device that includes laser, imaging, detection, and ranging (LIDAR) or other image-based processing capabilities such that a 3D view of the tissue site can be created from a scan made by the scanner 102. A device that includes LIDAR may utilize lasers to generate a signal corresponding to a topography of an object. For example, the device that includes LIDAR may determine a distance or a range by targeting a surface of an object with a laser and measuring an amount of time for reflected laser light to return to a receiver of the device. A device that includes LIDAR may be able to map the topography of an object with a high resolution which may create an accurate image of the surface of the object. Additionally or alternatively, the scanner 102 may be a device that has ultrasound or impedance capabilities.

The user interface 104 of the system 100 may be configured to receive input from a user. In some embodiments, the input from a user may relate to the tissue site to be scanned by the scanner 102 or may be input relating to another aspect of the system 100. For example, the user may identify a therapy to be applied to the tissue site and input the therapy information through the user interface 104. The therapy to be applied to the tissue site may be an input of the user interface 104 that identifies an intention for the tissue site or a therapy that is to be applied to the tissue site. For example, a user may be able to identify, through the user interface, that at least one of hydration therapy or oxygen therapy is to be applied to the tissue site. The user may have additional options such as bolstering therapy and/or closure therapy that a user may press to indicate an intention for the tissue site. Bolstering therapy may be selected if the tissue site needs additional structural support and closure therapy may be selected if the goal is to close the tissue site. Additionally or alternatively, the input from a user may be related to a type of tissue interface necessary to treat the tissue site. For example, the user may identify that the tissue interface should be a bolster, an all-around closure, a longitudinal closure, or a depth reduction tissue interface. In some embodiments, the user interface may be a screen that may be configured to display one or more options for the user to choose from. The user interface may be a touch screen surface in some embodiments. In other embodiments, the user interface may include one or more buttons to enable the user to select one or more options presented on the user interface.

In some embodiments, the scanner 102 may include additional components configured to communicate information about the system 100. For example, the scanner 102 may include one or more LEDs, one or more audio devices such as microphones and/or speakers, and one or more haptic elements. The one or more LEDs may be configured to display lights and/or images to convey information about the system 100. The one or more audio devices may enable the system to receive and output audio commands or information. The one or more haptic elements may be configured to vibrate to convey information about the system 100.

The processor 106 may be communicatively coupled with the scanner 102 and the user interface 104 and may be configured to receive the signal corresponding to the topography of the tissue site being scanned. The processor 106 may additionally be configured to receive the input from the user at the user interface. For example, the processor 106 may be configured to receive the user input identifying the therapy to be applied to the tissue site. The processor 106 may be hardware including logic circuits, a hardware/software combination that may be configured to execute software, or a combination thereof. For example, the processor 106 may include, but is not limited to, a central processing unit (CPU), an arithmetic logic unit (ALU), a digital signal processor, a microcomputer, a field programmable gate array (FPGA), a System-on-Chip (SoC), a programmable logic unit, a microprocessor, application-specific integrated circuit (ASIC), or another similar device.

The memory 108 may be coupled to the processor 106. The memory 108 may describe any of the terms “storage medium”, “computer readable storage medium” or “non-transitory computer readable storage medium” and may represent one or more devices for storing data, including read only memory (ROM), random access memory (RAM), magnetic RAM, core memory, magnetic disk storage mediums, optical storage mediums, flash memory devices and/or other tangible machine-readable mediums for storing information. The term “computer-readable medium” may include, but is not limited to, portable or fixed storage devices, optical storage devices, and various other mediums capable of storing, containing, or carrying instructions and/or data.

In some embodiments, the memory 108 and the processor 106 may both be components of a controller or a computer that may be configured to receive signals and execute instructions of the system 100. In some embodiments, the processor 106 may be configured as a special purpose machine (e.g., a processing device) to execute software or instructions, stored in the memory 108. The software may be embodied as program code including instructions for performing and/or controlling any or all operations described herein as being performed by the processor 106. More specifically, the memory 108 may be configured to store instructions that may be executed by the processor 106 in response to the processor 106 receiving the signal from the scanner 102. For example, the processor 106 may be configured to cause the system 100 to transform the signal corresponding to the topography of the tissue site into a model site surface and generate a model tissue interface based on the model site surface and the therapy to be applied to the tissue site. In some embodiments, the memory 108 may additionally be configured to store an image database 112 that may include images and/or information associated with different tissue sites. In some embodiments, generating the model tissue interface based on the model site surface and the therapy to be applied to the tissue site can include comparing the model site surface and the therapy to be applied to the tissue site to the images and information in the image database 112.

The output device 110 of the system 100 may be communicatively coupled to the processor 106. The output device 110 may be one or more devices configured to create the tissue interface based on the model tissue interface. For example, the output device 110 may be configured to receive the model tissue interface and, in response, generate a physical tissue interface representative of the model tissue interface. In some embodiments, the output device 110 may be a 3-dimensional (3-D) printer. In some embodiments, the output device 110 may be a fused deposition modeling 3-D printer. In some embodiments, the output device 110 may be a 3-D printer that may be configured to extrude the tissue interface into a coagulant bath. In some embodiments, the output device 110 may be a stereolithography printer. In some embodiments, the output device 110 may be a laser cutting device that may be configured to laser cut a wound filler material into the tissue interface. In some embodiments, the output device 110 may be a device that can output a template or information to allow a user to create a tissue interface based on the model tissue interface.

In some embodiments, the output device 110 may be a fused deposition modeling 3-D printer. The fused deposition modeling 3-D printer may print the tissue interface with bioabsorbable fibers. The bioabsorbable fibers may be fed from a spool of material through a moving, heated printer extruder head. The bioabsorbable fibers may then be deposited on a print bed in layers. There may be a plurality of layers needed to create the tissue interface. Once the tissue interface is printed, the tissue interface may be soaked in a bath to remove any printing support material created during the printing process. The printing support material may be used to support the tissue interface during the creation of the tissue interface but may not be a portion of the tissue interface. In some embodiments, soaking the tissue interface in a bath may include sterilizing in IPA or a similar substance.

In some embodiments, the output device 110 may be a 3-D printer that may be configured to extrude the tissue interface into a coagulant bath. Creating the tissue interface with a 3-D printer by extruding the tissue interface into a coagulant bath may include extruding or co-extruding the tissue interface material through an extrusion tip. The tissue interface material may be spun into a fiber by a spinneret and may be dispensed into a coagulant bath to create a solid membrane or matrix that may result in the tissue interface. In some embodiments, the tissue interface may be supported by a soluble support, or it may be self-supporting. The tissue interface may be sterile when it is formed as a result of printing into a closed environment.

In some embodiments, the output device 110 may be a stereolithography printer. Creating the tissue interface with the stereolithography printer may include using a computer-controlled moving laser beam. The laser beam may harden liquid resin that is contained in a reservoir to create a desired shape. The liquid resin may be a bioabsorbable material selected to form the tissue interface. The tissue interface may be sterile when it is created by printing into a closed environment. Additionally or alternatively, ultraviolet (UV) light may be used to cure the tissue interface and/or to sterilize the tissue interface.

In some embodiments, creating the tissue interface with the stereolithography printer may include stereolithography monochrome liquid crystal display (LCD) printing. The process of stereolithography monochrome LCD printing may use a high resolution, high contrast LCD panel. The LCD panel may be configured to harden liquid resin that is contained in a reservoir to create a desired shape. The liquid resin may be a bioabsorbable material selected to form the tissue interface. The LCD panel may use UV light emitting diodes (LEDs) to harden the liquid resin to cure the tissue interface to a high resolution. The tissue interface may be sterile when it is created by printing into a closed environment. Additionally or alternatively, ultraviolet (UV) light may be used to cure the tissue interface and/or to sterilize the tissue interface.

In some embodiments, the output device 110 may be a device that can transform bioabsorbable or other wound filler material into a customizable shape to fit the tissue site. For example, the output device 110 may be a laser cutting device that may be able to cut the tissue interface into a predetermined size and shape based on the model tissue interface.

In some embodiments, the output device 110 may be a device that can output a template or information to allow a user to create a tissue interface based on the model tissue interface. The template or information may enable the user to transform and customize bioabsorbable or other wound filler material into the tissue interface. For example, the user may be able to cut or trim the wound filler material to match the template or information output by the output device 110 such that the tissue interface is configured to fit into the tissue site.

The scanner 102, the user interface 104, and the output device 110 may be communicatively coupled to the processor 106. For example, the scanner 102, the user interface 104, and the output device 110 may be communicatively coupled through a Bluetooth connection, another wireless connection, or by a wired connection. In some embodiments, the scanner 102, the user interface 104, the output device 110, and the processor 106 may be separate devices that may be electrically or communicatively coupled to one another. In other embodiments, two or more of the scanner 102, the user interface 104, the output device 110, and/or the processor 106 may be included in one device. For example, in some embodiments, the scanner 102, the user interface 104, and the processor 106 may be included in one device such as a smart phone. The output device 110 may be configured to communicatively couple with the smart phone to create the tissue interface based on the model tissue interface.

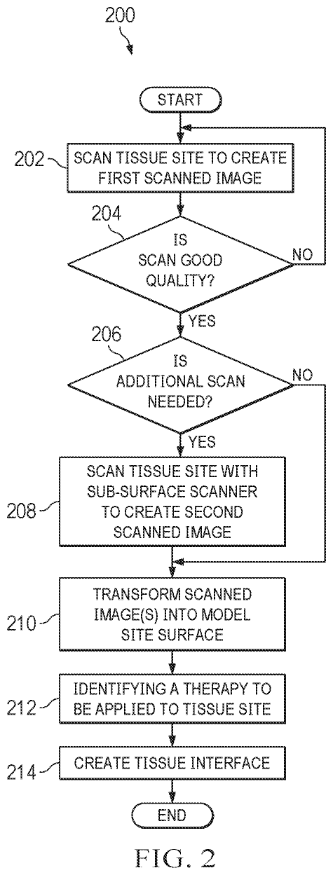

FIG. 2 is a flow chart 200 illustrating exemplary operations that may be associated with some embodiments of the system of FIG. 1. The process may start at block 202 where a tissue site is scanned to create a first scanned image. The first scanned image may be the scan created by the scanner when the tissue site is scanned. For example, the tissue site may be scanned by the scanner 102 of the system 100. The first scanned image may be the signal generated by the scanner 102 corresponding to the topography of the tissue site. At block 204, the process determines if the first scanned image is good quality. In some embodiments, the first scanned image may be considered good quality if the first scanned image has high resolution and does not contain gaps in surfaces of the first scanned image. In some embodiments, determining whether the first scanned image is good quality may include comparing the first scanned image to images stored in the image database 112. If the resolution of the first scanned image is within a range of resolutions of the images stored in the image database 112, the system 100 may determine that the first scanned image is good quality. The first scanned image may be considered poor quality if the system 100 does not determine that the first scanned image is good quality. For example, the processor 106 of the system 100 may be configured to analyze the first scanned image to determine whether the first scanned image may be considered good quality.

If the system 100 determines that the first scanned image is not good quality, the process continues on the NO path to block 202 where the process scans the tissue site to create the first scanned image. If the system 100 determines that the first scanned image is good quality, the process continues on the YES path to block 206.

In some embodiments, the user interface 104 may display a prompt indicating to the user that the first scanned image is good quality or poor quality. In other embodiments, the process may determine if the first scanned image is good quality and may proceed to either the block 202 or the block 206 automatically without an indication being output by the user interface 104. Additionally or alternatively, a user or a healthcare provider may be able to determine whether the first scanned image is good quality by viewing the first scanned image. The user may determine that the tissue site should be rescanned, and the user may scan the tissue site to recreate the first scanned image. In some embodiments, the user may be able to select an option on the user interface 104 selecting good quality or poor quality which may determine whether the process should follow the NO path to the block 202 or the YES path to the block 206.

At block 206, the process determines whether an additional scan is needed to create the model site surface. If an additional scan is needed, the process follows the YES path to block 208. There, the process scans the tissue site with a sub-surface scanner to create a second scanned image. In some embodiments, an additional scan may be needed if the tissue site includes sub-surface tunneling that is not captured by the first scanned image. More specifically, there may be features of the tissue site that may not be captured by the first scanned image. In some embodiments, the tissue site may have features that extend into the tissue site that may only be visible with a specialized scanner such as an ultrasound device. In some embodiments, a user or healthcare provider may be aware of subsurface features of the tissue site based on medical history of the patient or by features of the first scanned image and may select an option on the user interface 104 to force the process to follow the YES path to the block 208.

At the block 208, the tissue site may be scanned with the scanner 102 to create a second scanned image. In some embodiments, the scanner 102 may have ultrasound or impedance scanning capabilities to capture the sub-surface tunneling not captured by the first scanned image. In some embodiments, a second scanner with different capabilities that the scanner 102 may be used at the block 208 to scan the tissue site to create the second scanned image. The second scanned image may be a scan that include the topography of the tissue site and may include additional feature not visible in the first scanned image. The second scanned image may provide a more accurate image of the tissue site which may enable the system 100 to create a tissue interface that is similar in both size and shape to the tissue site including any subsurface features.

If an additional scan is not needed, the process follows the NO path to block 210. At the block 210, the process transforms the scanned images into a model site surface. For example, the processor 106 may use the first scanned image and the second scanned image to create the model site surface. In embodiments where the process does not create a second scanned image, the processor 106 may use the first scanned image to create the model site surface. The system 100 may create the model site surface by aligning visible and non-visible geometry of the tissue site as captured by one or both of the first scanned image and the second scanned image. The model site surface may include a 3-D image of the tissue site including all outer surfaces and inner surfaces as captured by one or both of the first scanned image and the second scanned image.

The process continues at block 212 where a therapy to be applied to the tissue site is identified. In some embodiments, identifying a therapy to be applied to a tissue site. In some embodiments, the therapy to be applied to the tissue site may be identified by receiving input from a user at the user interface 104. As discussed above, the therapy to be applied to the tissue site may be at least one of hydration therapy, oxygen therapy, bolstering therapy, and/or closure therapy. In some embodiments, the user may select one of “hydration therapy,” “oxygen therapy,” “bolstering therapy,” or “closure therapy” from the user interface 104 to identify either the therapy to be applied to the tissue site or the intention for the tissue site. In some embodiments, “hydration therapy” may be selected if the user intends for the tissue site to be hydrated. Hydration may prevent infection in some instances so “hydration therapy” may be selected if the tissue site is prone to infection. In some embodiments, “oxygen therapy” may be selected if additional oxygen may be beneficial to the wound. “Bolstering therapy” may be selected if the tissue site needs additional structural support and “closure therapy” may be selected if the goal is to close the tissue site. In some embodiments, treating a tissue site may require several dressing changes where different tissue interfaces are used. The therapy to be applied to the tissue site or the intention for treating the tissue site may be modified with each dressing change. For example, a first goal may be hydration so “hydration therapy” may be selected for a first dressing used to treat the tissue site. When it is time to change the dressing, a new therapy or intention may be selected such as “closure therapy” if the next goal is to close the tissue site. In some embodiments, the therapy options may be selected by a user on the user interface 104. In some embodiments, there may be additional options that may be selected and/or the options may be customized depending on the particular tissue site.

The process continues at block 214 where the tissue interface is created and the process ends. The tissue interface may be created by the output device 110 as describes above with reference to FIG. 1. The tissue interface may be created based on a model tissue interface created by the system 100 from both the model surface site and the identified therapy to be applied to the tissue site. In some embodiments, the output device 110 that is used to create the tissue interface may be chosen based on the model tissue interface and the identified therapy to be applied to the tissue site. In some embodiments, the internal structure of the tissue interface may be dependent on the identified therapy to be applied to the tissue site. For example, the internal structure of the tissue interface may include internal voids to allow one or both of lateral movement and horizontal movement depending on the identified therapy to be applied to the tissue site. In some embodiments, the tissue interface may be created with structures such as projections or cavities to aid in the therapy to be applied at the tissue site.

In some embodiments, there may be additional post process operations. For example, the tissue interface may be packaged for shipping or storage. In other embodiments, the tissue interface may be directly placed at the tissue site for the provision of therapy to the tissue site.

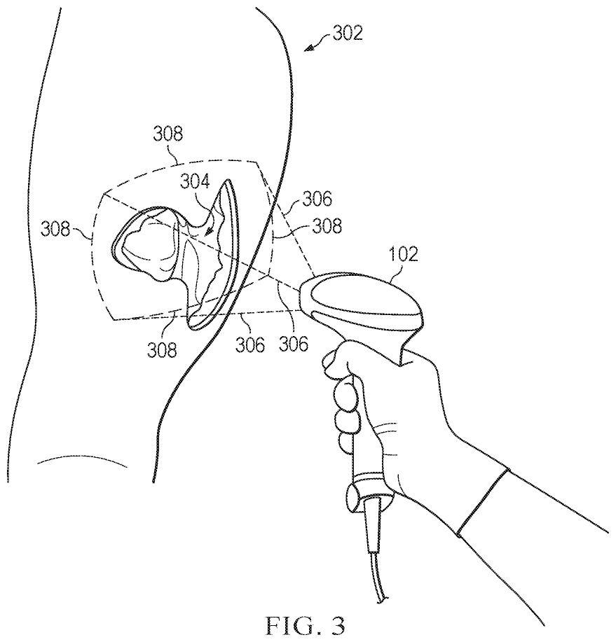

FIG. 3 is a perspective view of a tissue site 302 being scanned by the scanner 102, illustrating additional details that may be associated with some embodiments. The tissue site 302 may include a wound 304 that may be treated with a tissue interface. The scanner 102 may be equipped with LIDAR or another other image-based processing capabilities. In some embodiments, the scanner 102 may be an ultrasound device that may be configured to capture sub-surface features of the wound 304. Dashed lines 306 show a path where electromagnetic radiation such as light from a laser may travel from the scanner 102 to the tissue site 302. Dashed lines 308 may show an area on the tissue site 302 that may be scanned by the scanner 102. As shown in FIG. 3, the wound 304 may be larger than an area encompassed by dashed lines 308 and the scanner 102 may need to be adjusted to capture the wound 304 in its entirety. The scanner 102 may be configured to generate a signal corresponding to a topography of the tissue site 302.

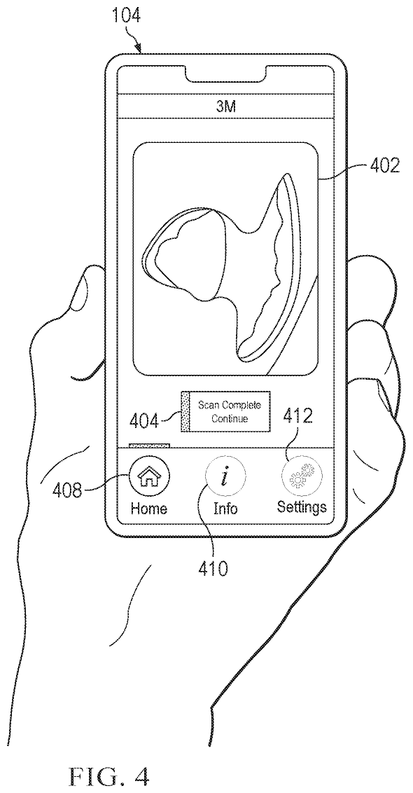

FIG. 4 is a schematic representation of a user interface displaying of a scanned image of the tissue site of FIG. 3. As shown in FIG. 4, the user interface 104 displays a scanned image 402 of the tissue site 302. The scanned image 402 may be generated based on the signal corresponding to the topography of the tissue site 302 as generated by the scanner 102. More specifically, the scanned image 402 may be a 3-D model of the tissue site 302. The user interface 104 may additionally include a quality indication icon 404. In some embodiments, the quality indication icon 404 may be a button or icon that a user can interact with on the user interface 104. As shown in FIG. 4, the quality indication icon 404 states “Scan Complete Continue.” The statement “Scan Complete Continue” can indicate to a user that the scanned image 402 is good quality and the tissue site 302 does not need to be re-scanned.

In some embodiments, the user interface 104 may additionally include a home icon 408, an info icon 410, and a settings icon 412. Interaction with the home icon 408, for example by pressing home icon 408, may cause the user interface 104 to display a home screen. In some embodiments, the home screen may not include the scanned image 402. Interaction with the info icon 410, for example by pressing the info icon 410, may cause the user interface 104 to display an informational page that may present information about the system 100 to the user. Interaction with the settings icon 412, for example by pressing the settings icon 412, may cause the user interface 104 to display a settings screen that may present the user different settings for one or more of the user interface 104 and the system 100.

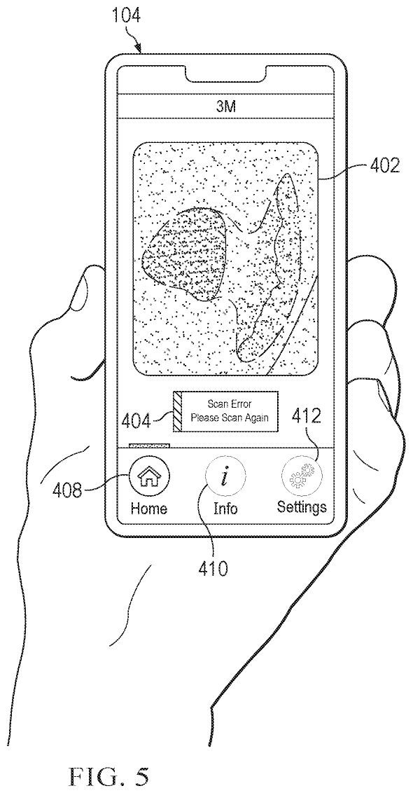

FIG. 5 is a schematic representation of the user interface displaying another scanned image of the tissue site of FIG. 3. As shown in FIG. 5, the scanned image 402 appears grainy and out of focus. The quality indication icon 404 states “Scan Error Please Scan Again.” The statement “Scan Error Please Scan Again” may indicate to a user that the scanned image 402 is poor quality and the tissue site 302 needs to be re-scanned for the system 100 to create the tissue interface. In some embodiments, the “Scan Error Please Scan Again” quality indication icon 404 may indicate that the scanned image 402 does not include a complete surface. In other embodiments, the “Scan Error Please Scan Again” quality indication icon 404 may indicate that the quality of the scanned image 402 is not high enough for the system 100 to create the model site surface from the scanned image.

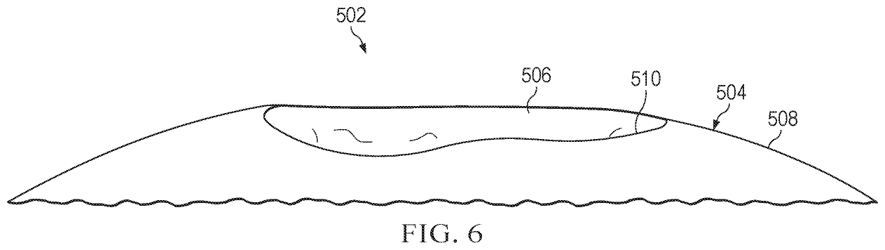

FIG. 6 is a side view of an exterior surface of a model site surface 502 generated from the scanned image 402 of FIG. 4. More specifically, a side view of an exterior surface 504 of the model site surface 502 is shown. The exterior surface 504 may include a wound portion 506 and a tissue site portion 508. The tissue site portion 508 may surround the wound portion 506. The tissue site portion 508 may define an outer boundary 510 of the wound portion 506. The model site surface 502 may be used to generate a model tissue interface for the wound 304 of the tissue site 302. The model tissue interface may be configured to be placed within the wound 304 of the tissue site 302 and may correspond to the wound portion 506 of the model site surface. The tissue site portion 508 of the model site surface 502 may be used to define outer boundaries of the model tissue interface.

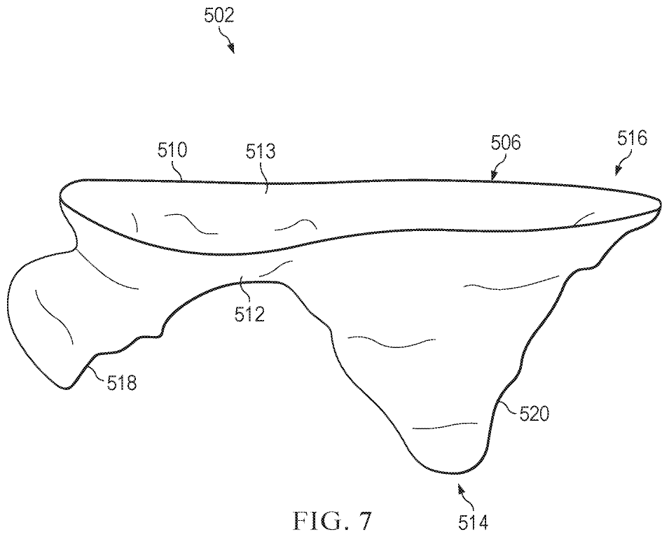

FIG. 7 is a perspective view of an interior surface of the model site surface 502 generated from the scanned image 402 of FIG. 4. In FIG. 7, an internal portion 512 of the wound portion 506 of the model site surface 502 is shown. The internal portion 512 of the model site surface 502 may correspond to a topography of the wound 304 of the tissue site 302 and may include an interior surface 513 that may define an interior surface of the wound 304 of the tissue site 302. The internal portion 512 may have a first end 514 and a second end 516 opposite the first end 514. The first end 514 may be a portion of the model site surface 502 that defines the deepest portions of the wound 304. The second end 516 may include the outer boundary 510 and may define a portion of the wound at a surface of the tissue site 302. In some embodiments, the wound 304 may include one or more deep portions which extend into the tissue site 302. The model site surface 502 may include a first extension 518 and a second extension 520 that may correspond to the one or more deep portions of the wound 304.

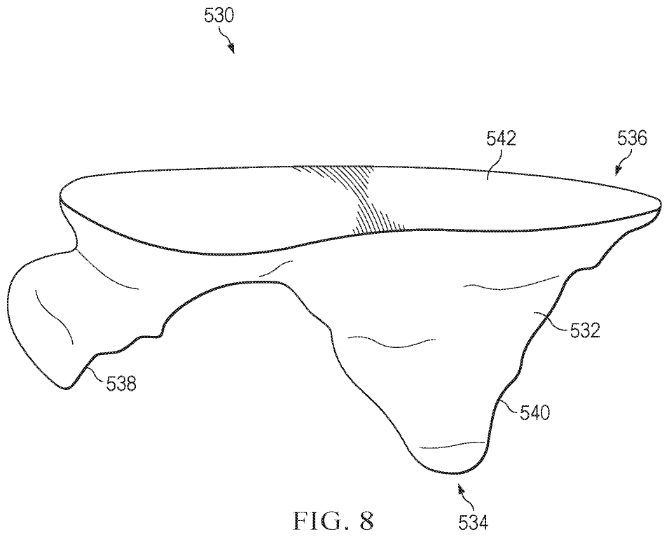

FIG. 8 is a perspective view of a model tissue interface generated from the model site surface of FIG. 6 and FIG. 7. In FIG. 8, a model tissue interface 530 that may be created based on the model site surface 502 is shown. The model tissue interface 530 may include an exterior 532 that may correspond to the topography of the wound 304 of the tissue site 302. The model tissue interface 530 may include a first end 534 and a second end 536 opposite the first end 534. The first end 534 may include one or more projections such as a first projection 538 and a second projection 540 corresponding to the first extension 518 and the second extension 520 of the model site surface 502. The model tissue interface 530 may also include a top surface 542. The top surface 542 of the model tissue interface 530 may be used to create a top surface of a tissue interface and may be configured to receive an interface or a port to couple the tissue interface to a negative pressure source.

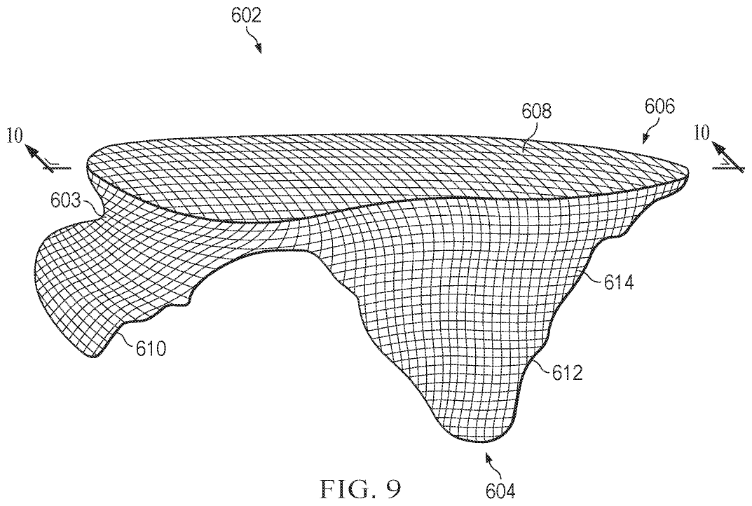

FIG. 9 is a perspective view of a tissue interface created based on the wound. A tissue interface 602 that may be created based on the model tissue interface 530. The tissue interface 602 may be substantially the same shape and size as the model tissue interface 530 and may include an outer surface 603 that may correspond to the topography of the wound 304 of the tissue site 302. The tissue interface 602 may include a first end 604 that may be configured to be disposed within the wound 304 of the tissue site 302. The tissue interface may include a second end 606 opposite the first end 604 that may be configured to be placed proximate to a periwound region of the tissue site 302 surrounding the wound 304. The second end 606 may include a top surface 608 that may be configured to receive an interface or a port to couple the tissue interface 602 to a negative pressure source. Additionally, the tissue interface 602 may include a first projection 610 that may correspond to the first projection 538 of the model tissue interface 530. The tissue interface 602 may additionally include a second projection 612 that may correspond to the second projection 540 of the model tissue interface 530.

In some embodiments, the tissue interface 602 may be formed from wound filler material 614 such as bioabsorbable fibers. In some embodiments, the wound filler material 614 of the outer surface 603 may interwoven to form a mesh pattern that may be dense and help to reduce tissue ingrowth and facilitate easy removal from the wound 304 of the tissue site 302.

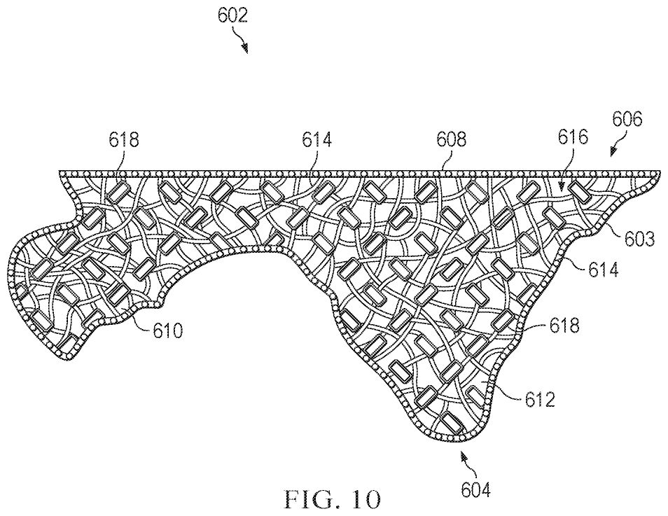

FIG. 10 is a cross-sectional view of the tissue interface of FIG. 9 taken along line 10-10 of FIG. 9. The wound filler material 614 may be disposed throughout an interior 616 of the tissue interface 602. There may be one or more internal voids 618 created or printed into the interior 616 of the tissue interface 602. The one or more internal voids 618 may be configured to allow lateral and/or vertical movement of the tissue interface 602. The tissue interface 602 may be configured to move laterally and/or vertically while the wound 304 is healing and in response to a force being applied to the tissue site 302 such as when a negative pressure source is actuated to apply negative pressure to the tissue site 302. Additionally, the internal voids 618 of the interior 616 of the tissue interface 602 may enable pressure at different points throughout the tissue interface 602 to be measured. The internal voids 618 of the interior 616 of the tissue interface 602 may be printed or created at desired locations in order to optimize negative pressure or instillation therapy being applied to the tissue site. For example, the internal voids 618 may be configured to distribute fluid to predetermined locations of the tissue site such as specific locations of the tissue site or the entire tissue site.

FIG. 11 is a cross-sectional view of the tissue interface 602 of FIG. 9 taken along line 10-10 of FIG. 9. In some embodiments, the tissue interface 602 may include a connection aperture 620 through the top surface 608 of the tissue interface 602. The connection aperture 620 may be configured to receive a pad, a port, or another element configured to couple the tissue interface 602 to a negative pressure source. In some embodiments, the system 100 may determine the optimal location for the connection aperture 620 based on the wound 304. The location of the connection aperture 620 may be determined by the system 100 based on any user input received and based on the topography of the wound 304 of the tissue site 302. The location of the connection aperture 620 may additionally be determined such that pathways through the interior 616 of the tissue interface 602 remain open and such that there is a proper seal between any pad, port, or other element configured to couple with the tissue interface 602 and the tissue interface 602. When the output device 110 is any of the 3-D printers described above with reference to FIG. 2, the connection aperture 620 may be printed into the tissue interface 602 while the tissue interface 602 is being printed by the 3-D printer.

In some embodiments, not shown, a connection pad may be formed integrally with the tissue interface 602. The connection pad may be a dressing interface, a pad, a port, or another element configured to couple the tissue interface 602 to a negative pressure source. The connection pad may be positioned at the connection aperture 620. If the output device 110 is any of the 3-D printers described above with reference to FIG. 2, the connection aperture 620, including the connection pad, may be printed into the tissue interface 602 during formation of the tissue interface 602 by the 3-D printer. If the connection pad is printed with the tissue interface 602, there may be fewer components to be arranged for the tissue interface 602 to be used in negative-pressure therapy. Therefore, arranging the tissue interface 602 at a tissue site and coupling the tissue interface 602 to a negative-pressure source to facilitate negative pressure therapy may be easier for a user.

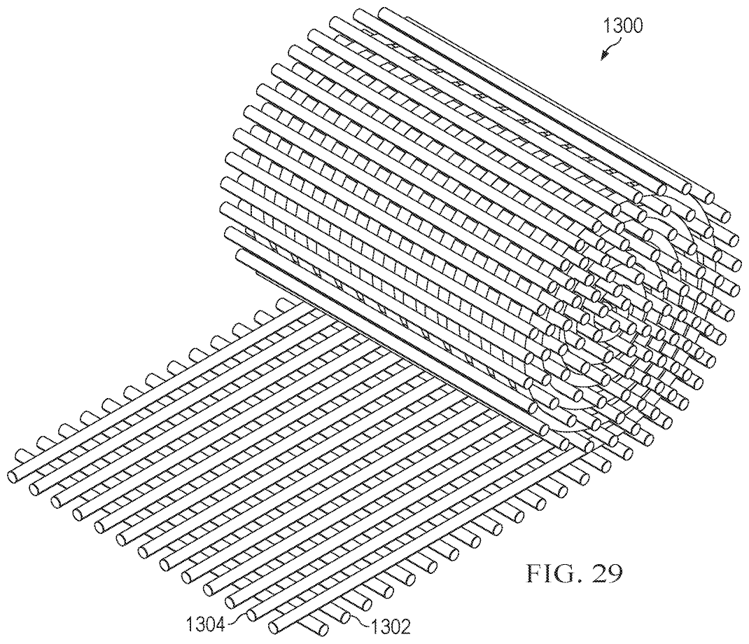

FIG. 12 thru FIG. 29 illustrate example embodiments of tissue interfaces that may be printed by any of the 3-D printing methods described above with reference to FIG. 2. Any of the tissue interfaces of FIG. 12 thru FIG. 29 may be formed from bioabsorbable polymers. In some embodiments, the bioabsorbable polymers may be polylactic acid or polylactides (PLA), polycaprolactone (PCL), polyglycolic acid or polyglycolides (PGA), polydioxanone (PDO), poly(glycerolsebacate) (PGS). In some embodiments, any of the above listed bioabsorbable polymers may be copolymerized or blended with one another and/or with glycerin and polyethylene glycols (PEG) to create a desired stiffness, elasticity, strength, and bioabsorption time for the tissue interface. In some embodiments, the tissue interface may additionally be modified by foaming plastics as the tissue interface is formed and to enhance stiffness and to control the amount of plastic used to create the tissue interface.

FIG. 12 is a cross-sectional view of a tissue interface 700 that may be created with a 3-D printer. The tissue interface 700 may include an exterior surface 702 and an interior 704 that may be surrounded by the exterior surface 702. In some embodiments, the exterior surface 702 may be an outer section and the interior 704 may be an inner section that may be surrounded by the outer section. The tissue interface 700 may be formed from fibers 706 of any of the above-described materials. The fibers 706 may be configured such that the exterior surface 702 has a density that is greater than the density of the interior 704. More specifically, the fibers 706 of the interior 704 may form an internal web that may have a density less than the density of the exterior surface 702. The density of the exterior surface 702 may additionally prevent tissue ingrowth into the tissue interface 700 and may make the tissue interface 700 easily removable from the tissue site 302. The structure of the tissue interface 700 may also enable the tissue interface 700 to remain at a tissue site for an extended period of time such as longer than three days.

The fibers 706 may be printed to create one or more internal voids 708. The one or more internal voids 708 may be configured to allow lateral movement of the tissue interface 700. Arrows 710 may indicate a direction of compression of the of the tissue interface 720 when a force is applied to the tissue interface 700. The tissue interface 700 may be configured to compress in the direction of the arrows 710 based on the placement of the internal voids 708.

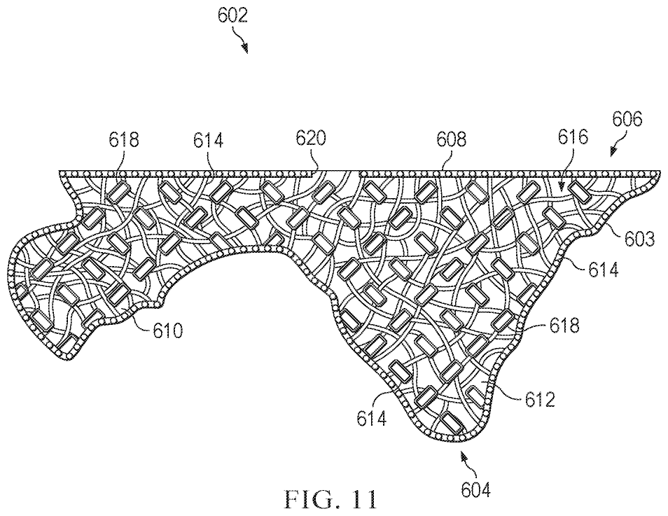

FIG. 13 is a cross-sectional view of a tissue interface 720 that may be created with a 3-D printer. The tissue interface 720 may have an exterior surface 722 and an interior 724 formed from fibers 726 substantially as described above with reference to the exterior surface 702, the interior 704, and the fibers 706 of FIG. 12. The fibers 726 may be printed in an internal web within the interior 724 to create one or more internal voids 728 that may be configured to allow vertical movement of the tissue interface 720. Arrows 730 may indicate a direction of compression of the tissue interface 720 when a force is applied to the tissue interface 720. The tissue interface 720 may be configured to compress in the direction of the arrows 730 based on the placement of the internal voids 728.

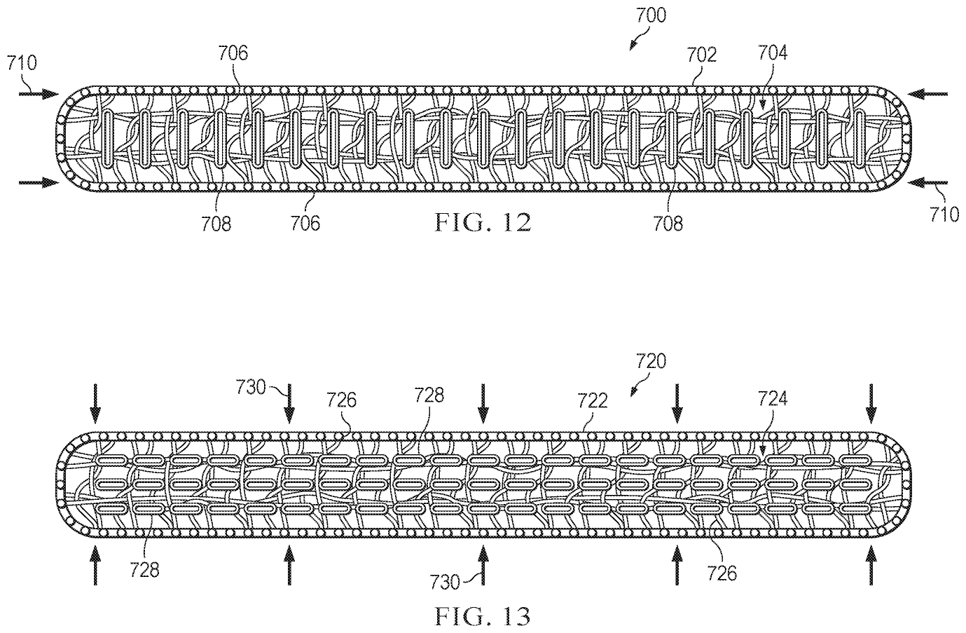

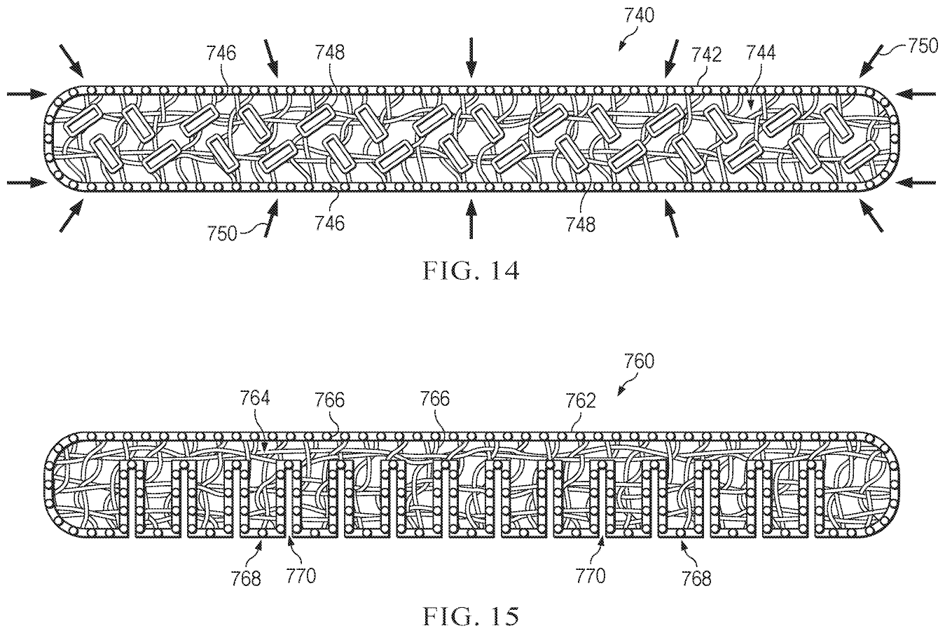

FIG. 14 is a cross-sectional view of a tissue interface 740 that may be created with a 3-D printer. The tissue interface 740 may have an exterior surface 742 and an interior 744 formed from fibers 746 substantially as described above with reference to the exterior surface 702, the interior 704, and the fibers 706 of FIG. 12. The fibers 746 may be printed to create one or more internal voids 748 that may be configured to allow lateral and vertical movement of the tissue interface 740. Arrows 750 may indicate a direction of compression of the tissue interface 740 when a force is applied to the tissue interface 740. The tissue interface 740 may be configured to compress in the direction of the arrows 750 based on the placement of the internal voids 748.

FIG. 15 is a cross-sectional view of a tissue interface 760 that may be created with a 3-D printer. The tissue interface 760 may have an exterior surface 762 and an interior 764 formed from fibers 766 substantially as described above with reference to the exterior surface 702, the interior 704, and the fibers 706 of FIG. 12. The tissue interface 760 may include one or more projections 768 that may define one or more cavities 770 between the one or more projections 768. In some embodiments, the one or more projections 768 and the one or more cavities 770 may be configured to aid in specific therapy at the tissue site that the tissue interface 760 is applied to. For example, the one or more projections 768 and the one or more cavities 770 may be configured to aid in treating the tissue site with instillation therapy by improving the distribution of instillation fluid throughout the tissue site.

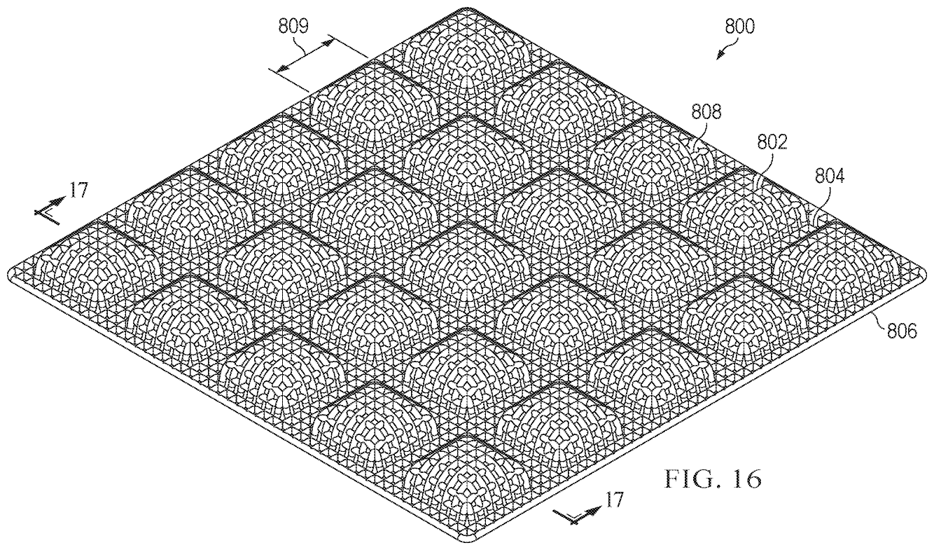

FIG. 16 is a perspective view of a tissue interface 800 that may be created with a 3-D printer. The tissue interface 800 may include one or more collapsible portions 802. The tissue interface 800 may additionally include a dense area 804 defining a periphery 806 of the tissue interface 800 and extending between the one or more collapsible portions 802. In some embodiments, the one or more collapsible portions may be at least one inner section and the dense area 804 may be an outer section that may have a greater density than the density of the at least one inner section. The one or more collapsible portions 802 may be configured to collapse when a force is applied to the tissue interface 800. The tissue interface 800 may be formed with fibers 808 of any of the above-described materials. The fibers 808 of the collapsible portions 802 may form an internal web that may have a density less than the density of the dense area 804. The density of the dense area 804 may prevent tissue ingrowth into the tissue interface 800 and may make the tissue interface 800 easily removable from the tissue site 302. The structure of the tissue interface 800 may also enable the tissue interface 800 to remain at a tissue site for an extended period of time such as longer than three days. In some embodiments, the one or more collapsible portions 802 may be substantially square and may have a length 809 of about two centimeters. In other embodiments, the one or more collapsible portions 802 may be a different size or shape that may be configured to collapse when a force is applied to the tissue interface 800.

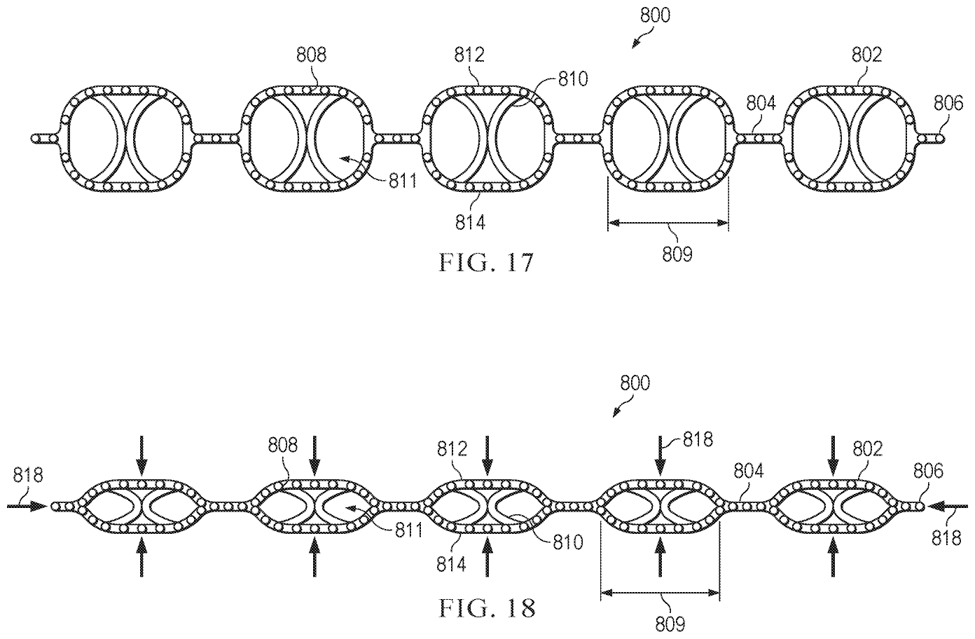

FIG. 17 is a cross-sectional view of an example embodiment of the tissue interface 800 of FIG. 16 taken along line 17-17 of FIG. 16. The one or more collapsible portions 802 may include internal fibers 810. The internal fibers 810 may form an internal web 811 and may extend from a top surface 812 of each of the collapsible portions 802 to a bottom surface 814 of each of the collapsible portions 802. The internal fibers 810 may allow for vertical collapse of the one or more collapsible portions 802 when a force is applied to the tissue interface 800. The internal fibers 810 may also allow for the one or more collapsible portions 802 to rebound back to an unstressed state after the force is removed from the tissue interface 800.

FIG. 18 is the cross-sectional view of the tissue interface 800 of FIG. 17 with a force applied to the tissue interface 800. Arrows 816 represent a vertical force being applied to the tissue interface 800 and arrows 818 represent a horizontal force being applied to the tissue interface 800. The tissue interface 800 may be compressed both horizontally and vertically due to the vertical force and the horizontal force. Upon removal of the vertical force and the horizontal force, the tissue interface 800 may rebound to the uncompressed position as shown in FIG. 16 due to the internal fibers 810.

FIG. 19 is a cross-sectional view of another embodiment of the tissue interface 800 taken along line 17-17 of FIG. 16. The tissue interface 800 may be substantially as described above with reference to FIG. 17 and FIG. 18 having the structure of the internal fibers 810 modified for different therapy intents. As shown in FIG. 19, the internal fibers 810 forming the internal web 811 may be coils or springs that may extend from the top surface 812 to the bottom surface 814 of each of the one or more collapsible portions. The internal fibers 810 may be configured to compress vertically with application of a vertical force and may enable each of the collapsible portions 802 to return to their uncompressed state after removal of the vertical force. Arrows 820 may indicate a direction of the vertical force that may be applied to the tissue interface 800.

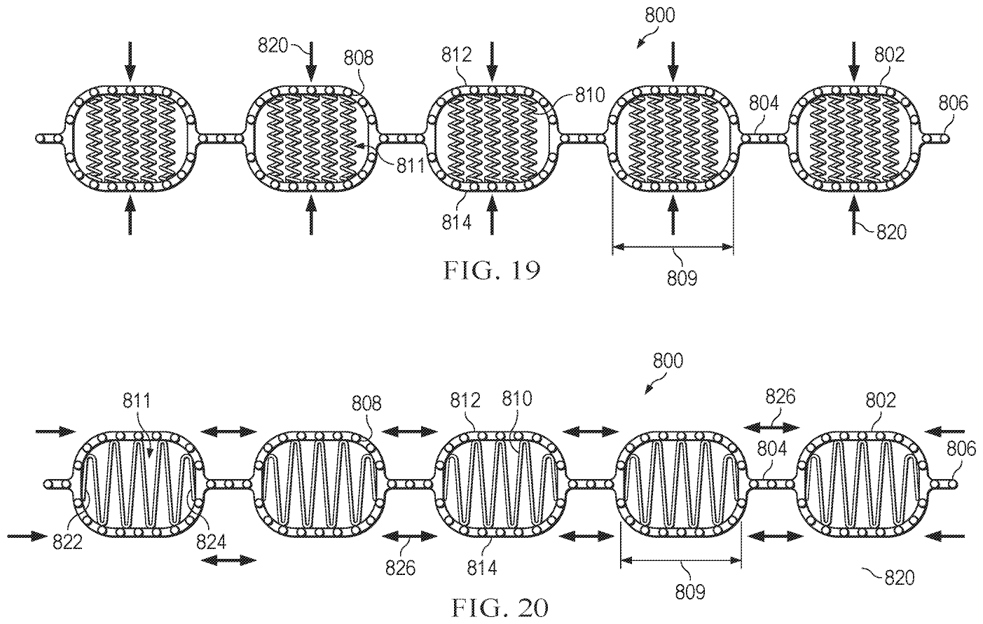

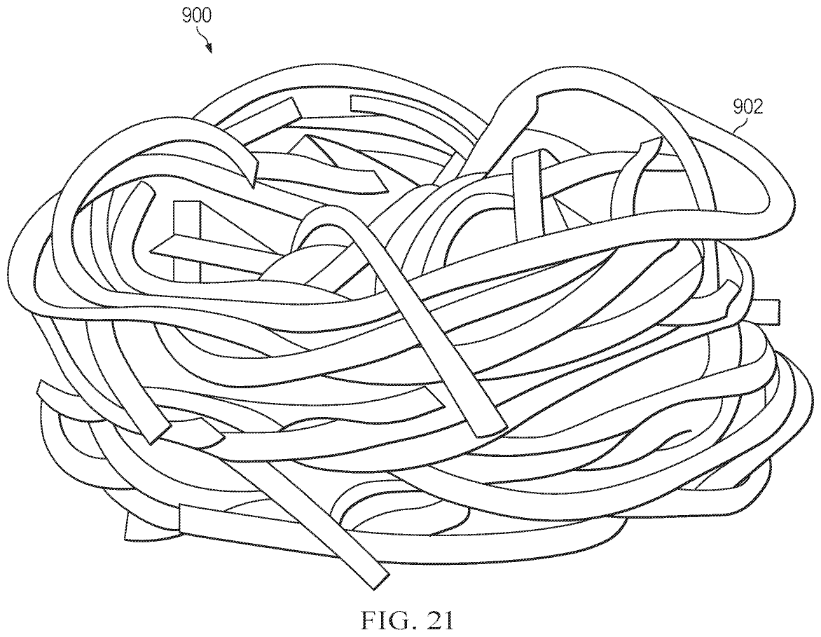

FIG. 20 is a cross-sectional view of another embodiment of the tissue interface 800 taken along line 17-17 of FIG. 16. The tissue interface 800 may be substantially as described above with reference to FIG. 17 and FIG. 18 having the structure of the internal fibers 810 modified for different therapy intents. As shown in FIG. 20, the internal fibers 810 forming the internal web 811 may be in the shape of a wave that may allow for lateral movement. Each of the internal fibers 810 may couple with the top surface 812 and the bottom surface 814 at several points from a first edge 822 to a second edge 824 of each of the collapsible portions 802. The internal fibers 810 may be configured to compress laterally with application of a lateral force and may enable each of the collapsible portions 802 to return to their uncompressed state after removal of the lateral force. Arrows 826 may indicate a direction of the lateral force that may be applied to the tissue interface 800.

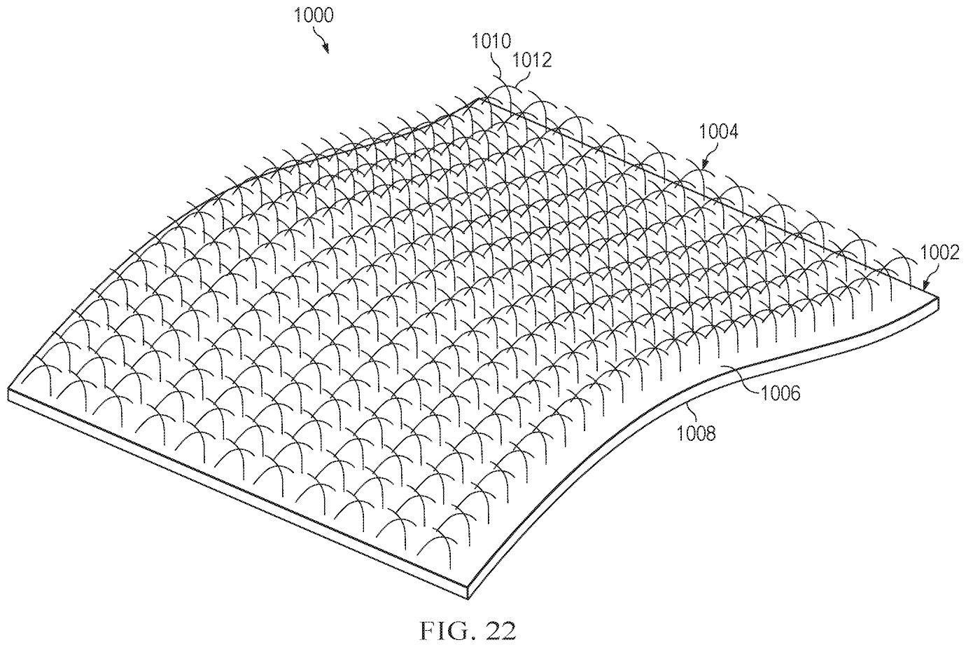

FIG. 21 is a perspective view of an example embodiment of a tissue interface 900. The tissue interface 900 may be formed from bioabsorbable wound filler material 902 of any of the specific materials described above. The bioabsorbable wound filler material 902 may be printed into a bundle. The bundle may be printed in a controlled arrangement such that the bioabsorbable wound filler material 902 allows negative pressure to be communicated through the tissue interface 900.

FIG. 22 is a perspective view of an example embodiment of a tissue interface 1000. The tissue interface 1000 may include a contact layer 1002 and a plurality of bioabsorbable hooks 1004 extending from a first surface 1006 of the contact layer 1002. In some embodiments, the contact layer 1002 may be configured to distribute fluid across the tissue site 302. The contact layer 1002 may be a woven sheet. In some embodiments, not pictured herein, the contact layer 1002 may be a perforated film that may include perforations or apertures to allow pressure communication through the contact layer 1002. The contact layer 1002 may additionally include a second surface 1008 opposite the first surface 1006. The contact layer 1002 may be configured to be positioned proximate to the tissue site 302. More specifically, the first surface 1006 may be positioned proximate to the tissue site 302 such that the plurality of bioabsorbable hooks 1004 may engage the tissue site 302.

The plurality of bioabsorbable hooks 1004 may extend across the first surface 1006 of the contact layer 1002. The plurality of bioabsorbable hooks 1004 may each include a first element 1010 and a second element 1012. The first element 1010 and the second element 1012 may be configured to couple and overlap to form each of the plurality of bioabsorbable hooks 1004. When the tissue interface 1000 is placed at the tissue site 302, the plurality of bioabsorbable hooks 1004 may engage with the tissue site 302. When the tissue interface 1000 is removed from the tissue site 302, the plurality of bioabsorbable hooks 1004 may break such that the first element 1010 is disengaged from the second element 1012 to provide easy removal from the tissue site 302.

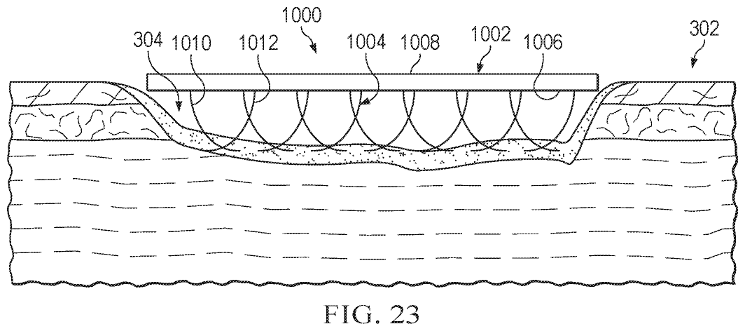

FIG. 23 is a schematic sectional view of the tissue interface 1000 placed within the wound 304 of the tissue site 302. The contact layer 1002 may be positioned such that the plurality of bioabsorbable hooks 1004 extend from the first surface 1006 of the contact layer 1002 towards the wound 304. The plurality of bioabsorbable hooks 1004 may be embedded within the wound 304 of the tissue site 302.

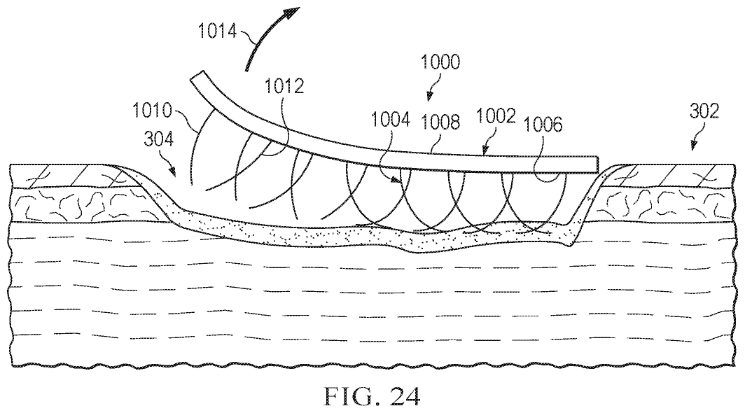

FIG. 24 is a schematic sectional view of the tissue interface 1000 being removed from the tissue site 302. Arrow 1014 may represent a force pulling the tissue interface 1000 away from the tissue site 302. The plurality of bioabsorbable hooks 1004 may break such that the first element 1010 disengages from the second element 1012 to facilitate removal of the tissue interface 1000 from the wound 304 of the tissue site 302. In some embodiments, the wound 304 may not grow into the plurality of bioabsorbable hooks 1004 and the plurality of bioabsorbable hooks 1004 may not break when the tissue interface 1000 is removed from the tissue site 302.

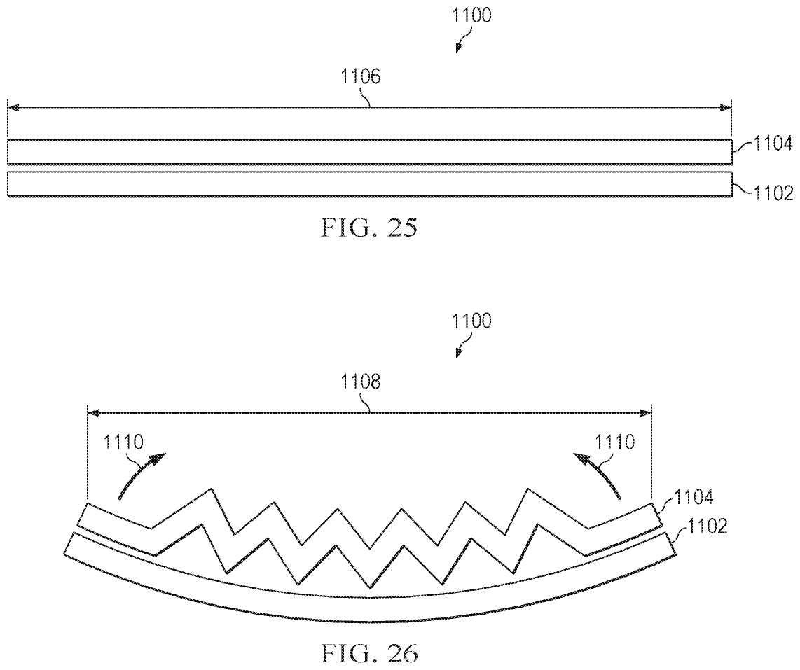

FIG. 25 is a side view of an example embodiment of a tissue interface 1100. The tissue interface 1100 may include a support layer 1102 and a transition layer 1104. The support layer 1102 may be configured to be positioned proximate to the tissue site 302. The transition layer 1104 may be coupled to the support layer 1102 and may have a dimension configured to transition between a first value and a second value in response to a change. For example, the dimension may be a length of the transition layer 1104 and the second value may be less than the first value such that the transition layer 1104 is configured to shrink in response to a change. The transition layer 1104 may have a length 1106 when in a resting state. In some embodiments, the dimension of the transition layer 1104 may transition between the first value and the second value in response to a change in temperature. Additionally or alternatively, the dimension of the transition layer 1104 may be configured to transition between the first value and the second value in response to exposure to a liquid.

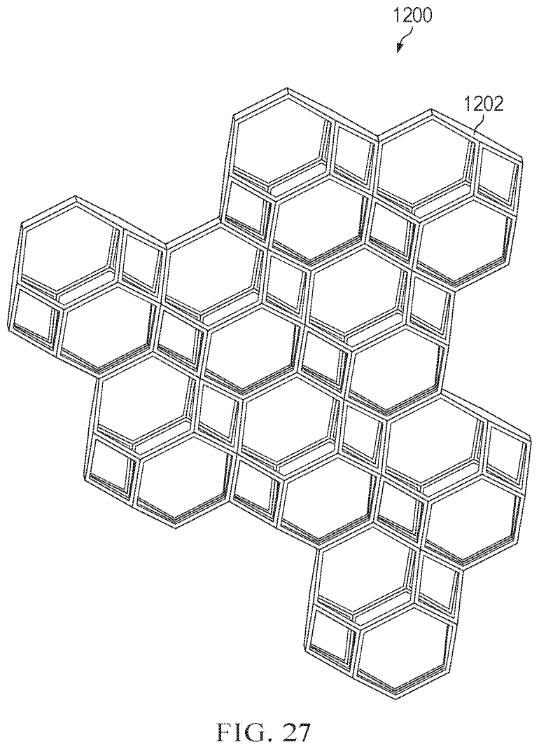

FIG. 26 is a side view of the tissue interface 1100 in a transitioned state. In the transitioned state, the tissue interface 1100 may be exposed either a change of temperature or may be exposed to a liquid which may cause the transition layer 1104 to undergo a change. In some embodiments, the transition layer 1104 may shrink to a transition length 1108 that may be less than the length 1106 when the tissue interface 1100 is in the resting state. The transition layer 1104 may fold to transform from the length 1106 to the transition length 1108. In some embodiments, arrows 1110 may represent a force applied to the support layer 1102 when the transition layer 1104 has the transition length 1108. When the force is applied to the support layer 1102, the support layer 1102 may bend. In some embodiments, the support layer 1102 may additionally form a mesh that may manifold pressure through the tissue interface 1100 when the transition layer 1104 has the transition length 1108.

The transition layer 1104 may be coextruded from filaments that may have one or more of heat shrink properties or wet shrink properties. The support layer 1102 may be formed from filaments that are more rigid than the filaments of the transition layer 1104 to provide support and structure to the tissue interface 1100. Thus, the support layer 1102 may have a rigidity that may be greater than a rigidity of the transition layer 1104.

FIG. 27 is a perspective view of an example embodiment of a tissue interface 1200. The tissue interface 1200 may be 3-D printed into a geometric mesh 1202. The geometric mesh 1202 may create support for the tissue interface 1200 and may enable pressure manifolding through the tissue interface 1200. The geometric mesh 1202 may additionally allow for rebound of the tissue interface 1200 which may further enable pressure manifolding. In some embodiments, the geometric mesh 1202 of the tissue interface 1200 is formed from truncated octahedrons. Other geometric mesh shapes may achieve the goals of support and pressure manifolding of the tissue interface 1200.

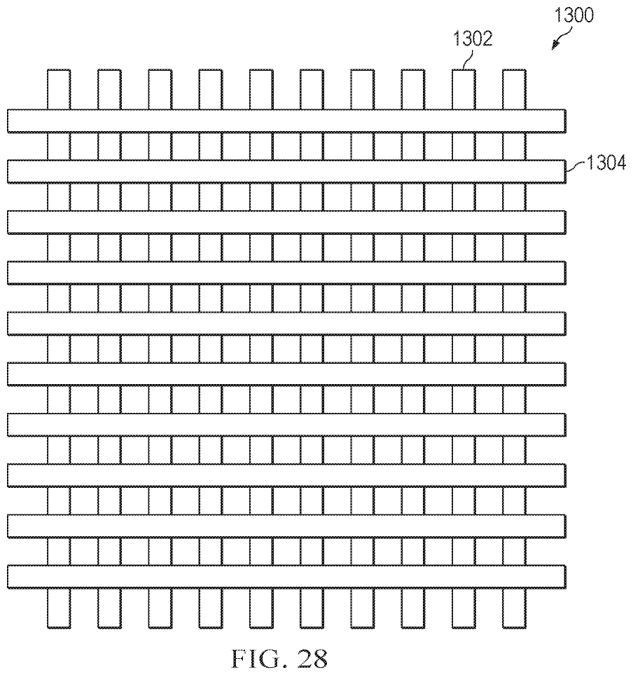

FIG. 28 is a top view of an example embodiment of a tissue interface 1300. The tissue interface 1300 may be a lattice of fibers that may be able to be applied similar to how gauze is applied to a patient. The tissue interface 1300 may include a first plurality of fibers 1302 that may be oriented in a first linear orientation. The tissue interface 1300 may additionally include a second plurality of fibers 1304 that may be oriented in a second linear orientation. In some embodiments, the second linear orientation may be perpendicular to the first linear orientation. The tissue interface 1300 may be created by 3-D printing the first plurality of fibers 1302 in the first linear orientation and then 3-D printing the second plurality of fibers 1304 in the second linear orientation. In some embodiments, the tissue interface 1300 may include additional layers. For example, not pictured herein, the tissue interface 1300 may include a third plurality of fibers that may be printed onto the second plurality of fibers 1304. The third plurality of fibers may be oriented in the first linear orientation such that they are oriented perpendicular to the second plurality of fibers 1304. In some embodiments, additional layers may be printed to the tissue interface 1300 until a desired thickness is acquired. In some embodiments, the tissue interface 1300 may be between about 5 millimeters to about 20 millimeters thick.

FIG. 29 is a perspective view of the tissue interface 1300. The tissue interface 1300 may be rolled so that it can be applied to the tissue site 302 similar to gauze. The tissue interface 1300 being rolled may make it easier to apply to the tissue site 302 and may male it easier to store and/or transport.

The systems, apparatuses, and methods described herein may provide significant advantages. For example, the system 100 may be configured to create the tissue interface 602 with reduced waste and the tissue interface 602 may have a longer wear time, improved usability, and less risk of any of the wound filler material 614 remaining in the wound 304 due to the tissue interface 602 being customized to the wound 304. The system 100 may enable the tissue interface 602 to be customized to the wound 304 of a tissue site 302 which may enable the tissue interface 602 to be optimized for the therapy to be applied to the tissue site 302. It may also be advantageous that the system 100 can optimize the location of the connection aperture 620 on the tissue interface 602. The tissue interface 602 may additionally provide advantages such as being comprised of bioabsorbable material and reducing trauma from removal and reducing the risk of tissue ingrowth due to the outer surface 603 being dense. The tissue interface 602 may also enable more efficient wound packing of the wound 304 which may reduce the time required by a caregiver to treat the wound 304.

The tissue interfaces of FIGS. 12-29 may also provide significant advantages. For example, the tissue interfaces may be 3-D printed from bioabsorbable materials. The tissue interfaces may be constructed of a reduced amount of material and may provide structure and support to the tissue site 302 while enabling the manifolding of negative pressure through the tissue interface. Additionally or alternatively, the tissue interface may be configured to collapse in at least a vertical or a horizontal direction to provide different levels of compression for a given tissue site.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

1. A system for creating a tissue interface, the system comprising:
a scanner configured to scan a tissue site and generate a signal corresponding to a topography of the tissue site;
at least one processor configured to:
receive the signal corresponding to the topography of the tissue site and receive user input at a user interface identifying a therapy to be applied to the tissue site; and
a memory coupled to the at least one processor configured to store instructions that when executed by the at least one processor in response to receiving the signal corresponding to the topography of the tissue site cause the system to:
transform the signal corresponding to the topography of the tissue site into a model site surface; and
generate a model tissue interface based on the model site surface and the therapy to be applied to the tissue site.

2. The system of claim 1, wherein the instructions that when executed by the at least one processor cause the system to create a tissue interface based on the model tissue interface.

3. The system of claim 2, wherein creating the tissue interface based on the model tissue interface comprises printing the tissue interface using fused deposition modeling.

4. The system of claim 3, wherein printing the tissue interface further comprises depositing a plurality of bioabsorbable fibers in a plurality of layers.

5. The system of claim 3, wherein creating the tissue interface based on the model tissue interface further comprising removing printing support material from the tissue interface.

6. (canceled)

7. (canceled)

8. The system of claim 2, wherein creating the tissue interface based on the model tissue interface comprises extruding a bioabsorbable material into a coagulation bath.

9. The system of claim 2, wherein creating the tissue interface based on the model tissue interface comprises printing the tissue interface using stereolithography.

10. The system of claim 9, wherein creating the tissue interface based on the model tissue interface further comprising curing and sterilizing the tissue interface by exposing the tissue interface to an ultraviolet (UV) electromagnetic radiation source.

11. (canceled)

12. The system of claim 2, wherein creating the tissue interface based on the model tissue interface comprises stereolithography monochrome liquid crystal display (LCD) printing.

13. (canceled)

14. (canceled)

15. The system of claim 2, wherein creating the tissue interface based on the model tissue interface comprises laser cutting the tissue interface to a predetermined size and shape based on the model tissue interface.

16. The system of claim 2, wherein creating the tissue interface based on the model tissue interface further comprises creating a connection pad formed integrally with the tissue interface.

17. The system of claim 2, wherein the tissue interface includes structures configured to distribute fluid to predetermined locations of the tissue site.

18. The system of claim 1, wherein the instructions that when executed by the at least one processor cause the system to create a template based on the model tissue interface, the template configured to enable a user to customize a tissue interface to fit the tissue site.

19. The system of claim 1, wherein the memory stores an image database including images and information associated with different tissue sites.

20. The system of claim 19, wherein generating the model tissue interface based on the model site surface and the therapy to be applied to the tissue site comprises comparing the model site surface and the therapy to be applied to the tissue site to the images and information in the image database.

21. A method of creating a tissue interface, the method comprising:
scanning a tissue site to create at least one scanned image;
transforming the at least one scanned image into a model site surface;
identifying a therapy to be applied to the tissue site; and
creating the tissue interface based on the model site surface and the therapy.

22. The method of claim 21, wherein scanning the tissue site comprises imaging the tissue site with laser imaging, detection, and ranging (LIDAR) technology and imaging the tissue site with an ultrasound device configured to capture sub-surface features.

23. (canceled)

24. The method of claim 21, wherein the at least one scanned image comprises a three-dimensional (3-D) model of the tissue site.

25. The method of claim 21, further comprising analyzing the at least one scanned image and if the at least one scanned image does not include a complete surface, generating an indicator to re-scan the tissue site.

26. The method of claim 21, wherein identifying the therapy to be applied to the tissue site comprises identifying at least one of hydration, oxygen, bolstering, and closure.

27-46. (canceled)
